(19) United States
(12) Patent Application Publication
(10) Pub. No.: US 2004/0116505 A1
(43) Pub. Date: Jun. 17, 2004

(54) Treatment of tics, tremors and related disorders

(76) Inventors: Gregory Krauss, Baltimore, MD (US); Harvey Singer, Baltimore, MD (US)

Correspondence Address: EDWARDS & ANGELL, LLP, P.O. BOX 55874, BOSTON, MA, 02205, US

(21) Appl. No.: 10/468,937

(22) PCT Filed: Feb. 22, 2002
(86) PCT No.: PCT/US02/05189

(30) Foreign Application Priority Data
Feb. 23, 2001 (US) 60270987

(51) Int. Cl. A61K 31/4015

(52) U.S. Cl. 514/424

(57) ABSTRACT

The present invention relates to methods for the treatment of patients suffering from or susceptible to a hyperkinetic movement disorder such as a tic or tremor. In preferred aspects, the invention provides methods for treatment of tic disorders including Tourette syndrome by administration of one or more pyrrolidone compounds possessing anti-convulsant activity.

The present application claims the benefit of U.S. provisional application No. 60/270,987 filed Feb. 23, 2001, which is incorporated herein by reference in its entirety.

### FIELD OF THE INVENTION

The invention provides new methods for treating a subject suffering from or susceptible to one or more disorders causing repetitive, involuntary movements or vocalizations including any combination of one or more hyperkinetic or hypokinetic movement disorders. Therapies of the invention include administration an anticonvulsant compound (i.e. a compound that can prevent or relieve convulsions) to a subject in need thereof, such as a subject suffering from or susceptible to one or more disorders such as tremor, dyskinesias, myoclonus, simple or complex tic, Tourette syndrome, or drug induced movement disorders.

### BACKGROUND OF THE INVENTION

Tics are common, affecting up to 1 percent of school-aged children, and are frequently socially disabling. Current drug therapies for tics are very limited either due to a lack of efficacy or to frequent and serious side effects.

Tic disorders, including simple motor, complex motor, complex vocal and Tourette syndrome, are common and often disabling neurological disorders. They are frequently associated with behavior difficulties, including obsessive-compulsive disorder, attention deficit hyperactivity disorder and impulse control. Current therapies for tics are limited, due to either frequent side-effects or limited efficacy. For example, neuroleptics are associated with sedation, weight gain and tardive diskinesia. Levetiracetam is a generally well-tolerated anticonvulsant that has been investigated as a therapeutic agent for the treatment of epileptic seizures.

Tics are easy to observe but hard to appreciate from a written description: they are involuntary, sudden, rapid, repetitive, nonrhythmic stereotyped movements or vocalizations. Tics are manifested in a variety of forms, with different durations and degrees of complexity, and no two patients have exactly the same symptoms. Simple motor tics are brief rapid movement s that often involve only one muscle group (e.g., eye blink, head jerk, shoulder shrug). Complex motor tics are abrupt movements that involve either a cluster of simple movements or a more coordinated sequence of movements. Complex motor tics may be non-purposeful (facial or body contortions) or appear to be more purposeful but actually serve no purpose (touching, smelling, jumping, obscene gestures).

Complex motor tics should be differentiated form stereotypes and compulsions. Stereotypic movements such as head banging and rocking, tend to be rhythmic and intentional, in contrast to tics which are non-ryhthmic and involuntarily. Touching and tapping may be either tics or compulsive symptoms: however, the latter usually occur in association with other obsessive-compulsive symptoms and may be preceded by a conscious need to perform the action to avoid an unwanted circumstance or a feeling.

Simple vocal tics include sounds such as grunting, barking, yelping, and throat clearing. Complex vocalization may include syllables, phrases, echolalia (repeating other people's words), palilalia (repeating one's own words), or coprolalia (obscene words). Tourette syndrome (TS) is an autosomal dominant multiple tic disorder with variable penetrance that begins in childhood, often with simple tics, and progresses to multiple, complex movements including respiratory and vocal tics. Tourette syndrome frequently includes one or more vocal tics which begin as grunting or barking noises and later develop into compulsive utterances.

Drug therapy is reserved for tics that are functionally disabling since none of the available pharmacotherapies for tics is curative and all are associated with potential harmful side effects. Patients with mild tics are counseled and observed for the progression of symptoms. In general the first line of pharmacotherapy in children with milder tics, especially in those with behavioral problems (i.e., inattentiveness, impulsivity, poor frustration tolerance, and aggressive outbursts, is clonidine. This alpha-2 adrenergic receptor agonist acts selectively at the presynaptic level when used in lower doses. The efficiency of clonidine for the treatment of tics, however, remains controversial (Goetz et al., 1987; Leckman et al., 1991). Neuroleptics are the most effective tic-suppressing agents, but side effects often limit their usefulness. Complications may occur even when low doses of drugs are prescribed. Although haloperidol was the first agent of this type used, in view of a higher frequency of serious side effects and significantly greater extrapyramidal symptoms many clinicians prefer starting with pimozide. Other neuroleptic agents with tic-suppressing capabilities but not approved by the FDA for Tourette syndrome include fluphenazine and risperidone. Botulinurn toxin injections have been used successfully in the treatment of dystonic and other tics.

Tremor is a rhythmic, alternating oscillatory movement produced by repetitive patterns of muscle contraction and relaxation. Tremors are classified according to their rate (slow −3 to 5 Hz; rapid 6 to 12 Hz), rhythm, distribution, and whether they occur at rest (Resting tremor) or during muscular activity (familial, action, or intention tremors. Tremors include physiologic tremors, enhanced physiologic tremors, benign hereditary tremors (essential tremors, resting tremors of Parkinson's disease, intention tremor of cerebellar disease, familial tremor, titubation, hepatolenticular degeneration, and asterixis. A number of therapeutic agents have been administered in the treatment of tremors such as benzodiazepine anziolytics such as diazepam, lorazepam or oxazepam, propranolol, primidone. However, each of them exhibits adverse side effects or is poorly tolerated by subjects dosed with sufficient therapeutic agent to treat the tremor.

Myoclonus is a brief, lightning-like contraction of a muscle or group of muscles which includes nocturnal myoclonus, singultus, e.g., common hiccup, etiology, action myoclonus, and palatal myoclonus. Many forms of myoclonus result from an underlyng metabolic disorder, closed head trauma, ischemic brain injury, or various degenerative diseases. Treatment of the underlying metabolic abnormalities is typically preferred. Alternatively clonazepam or valproic acid may be effective in treatment of patients suffering from myoclonus.

### SUMMARY OF THE INVENTION

We have surprisingly discovered that levetiracetam is effective for treating post-hypoxic and post-encephalitic myoclonus, tremor and tic disorders.

We now provide therapies for treatment of a subject suffering from or susceptible to hyperkinetic or hypokinetic movement disorders including such disorders which inflict the subject with repetitive involuntary movements or vocalizations. The present invention relates to methods of reducing the frequency, duration or severity of episodes of repetitive involuntary movements or vocalizations induced by a hyperkinetic or hypokinetic movement disorder which is suitable for treatment by the methods of the invention.

In preferred embodiments, the invention includes methods for the treatment of any disorder, which causes involuntary, repetitive movements or utterances by the administration of an anticonvulsant therapeutic agent to the subject suffering from or susceptible to a hyperkinetic, or hypokinetic movement disorder. Preferred anti-convulsant therapeutics suitable for use in the methods of the invention include those anticonvulsant compounds comprising a pyrrolidone functional group. Particularly preferred anticonvulsants include optionally substituted N-(2-acetamide)-2-pyrrolidone compounds. Particularly preferred anticonvulsants suitable for use in the methods of the invention include Levetiracetam and piracetam.

In general, the methods of the present invention are useful for treating a variety of movement disorders wherein the disorder involves a repetitive involuntary movements or vocalizations. Methods of the invention include those methods suitable for treatment of Tics, Tourette Syndrome, tremor, or myoclonus, more preferable are treatment methods suitable the treatment of tic disorders including simple tics, complex tics, and Tourette syndrome. Particularly preferred methods of the invention are suitable for the treatment of complex tics and Tourette syndrome without an adverse or harmful side effects. The treatment methods of the invention in general comprise administration of a therapeutically effective amount of one or more pyrrolidone compounds with anti-convulsant activity to a patient in need of treatment, such as a mammal, particularly a primate such as a human.

In other embodiments, the therapeutic methods of the invention are suitable for treating subjects who are suffering from or are susceptible to simple or complex tics, Tourette syndrome, or tremor and are non-responsive to other therapeutic regiments. The therapeutic methods of the invention are preferably suitable for subjects having manifestations or episodes of simple or complex tics, Tourette syndrome, tremor or other movement disorders which did not decrease in frequency, duration or severity upon administration of a standard therapeutic such as haloperidol, clonidine or a benzodiazepine anxiolytics.

The methods of the invention are suitable for treating subjects who failed to respond to treatment with one or more therapeutic agents commonly used in the treatment of tic disorders including Tourette syndrome. Administration of a pyrrolidone anti-convulsant agent to such a subject results in a reduction in the frequency, duration or severity of occurrences of the afflicting movement disorder.

Preferred methods of the invention including identifying and/or selecting a subject (e.g. mammal, particularly human) that is susceptible to or suffering from a condition disclosed herein, particularly a subject that is susceptible to or suffering from involuntary, repetitive movements or utterances, especially Tics, Tourette Syndrome, tremor, or myoclonus, even more preferably a subject that is susceptible to or suffering from tic disorders including simple tics, complex tics, and Tourette syndrome

Pyrrolidone anti-convulsant therapeutic agents suitable for use in the treatment methods of the invention preferably have minimal adverse side effects. More preferably such therapeutic agents exhibit no adverse side effects or minimal side effects in a small subset of the population such that the treatment methods of the invention are suitable for short-term and long-term administration to a patient. Particularly preferred therapeutic agents may be administered on a prophylactic basis.

Specifically preferred pyrrolidone anti-convulsant compounds for use in the methods of the invention include levetiracetam (((S)-2-(2-Oxo-pyrrolidin-1-yl)-butyrarnide), piracetam (2-(2-Oxo-pyrrolidin-1-yl)-acetamide), and pharmaceutically acceptable salts of those compounds.

The invention also provides pharmaceutical compositions that comprise one or more compounds of the invention and a suitable carrier for the compositions.

Other aspects of the invention are disclosed infra.

### DETAILED DESCRIPTION OF THE INVENTION

As stated above, and demonstrated in the examples which follow, it has now been found that administration of a pyrrolidone anti-convulsant compound to a subject can reduce the frequency, duration or severity of episodes of a hyperkinetic or hypokinetic movement disorder. Thus, administration of a pyrrolidone anti-convulsants can reduce the occurrence of movement disorders, such as tics, tremors, myoclonus,

It is believed the methods of the invention are further unique in that they are suitable for treating a variety of movement disorders such as simple tics, complex tics, Tourette syndrome, and tremors induced by any one of a variety of underlying disorders. Moreover, the methods of the invention are unique in that they are suitable for treatment of patients who are non-responsive to other therapeutic methods and therapeutic agents.

The methods of the invention in general comprise administration of a therapeutically effective amount of one or more pyrrolidone anticonvulsant compounds to a patient in need of treatment. Typical subjects for treatment include persons susceptible to, suffering from or that have suffered a hyperkinetic movement disorder or a hypokinetic movement disorder. In particular, suitable subjects for treatment in accordance with the invention include persons that are susceptible to, suffering from or that have suffered (a) tremors, including physiologic tremors, benign hereditary tremors, resting tremors associated with Parkinson's disease or other neurologic diseases, and intention tremors associated with cerebellar diseases; (b) tics, including simple tics, complex tics, and multiple tic disorders such as Tourette syndrome; (c) myoclonus; or (d) Dystonia, including generalized dystonia, segmental dystonia or focal dystonia. Particularly preferred subjects for treatment in accordance with the invention include persons that are susceptible to, suffering from or that have suffered tremors which optionally may be induced by an underlying condition or disorder, simple tics, complex tics comprising involuntary movement, vocalization or both, and Tourette syndrome.

The invention also provides methods for treatment of a subject suffering from tic disorders including simple tics and complex tics comprising the administration of a pyrrolidone compound having anticonvulsant activity to a subject suffering from a tic disorder. Preferred compounds suitable for use in treating subjects suffering from tic disorders include compounds of any one of Formulae I, II or III, more preferred compounds are levetiracetam and piracetam.

Methods of the invention which are suitable for treatment of tic disorders are preferably also suitable for treatment of Tourette syndrome and other multiple tic disorders.

The invention further provides a method treatment of a subject suffering from a tremor disorder including resting tremors, intention tremors and familial tremors comprising the administration of a pyrrolidone compound having anticonvulsant activity to a subject suffering from a tremor disorder. Preferred compounds suitable for use in treating subjects suffering from tremor disorders include compounds of any one of Formulae I, II or III, more preferred compounds are levetiracetam and piracetam.

Methods of the invention which are suitable for treatment of tremor disorders are preferably also suitable for treatment of tremors associated with neurodegenerative diseases such as Parkinson's disease or cerebellar diseases such as multiple sclerosis and other cerebellar outflow diseases.

The methods of the invention for the treatment of tic disorders, tremors, and Tourette syndrome comprise administration of an effective amount of one or more compounds of the invention to a patient in need of treatment.

Pyrrolidone compounds suitable for use in the methods of the invention are typically well tolerated, are efficiently take up in the body by a suitable mode of administration commonly used for treatment of hyperkinetic movement disorders such as tics, tremors and the like and have few cognitive side effects. In a non-limiting example, levetiracetam exhibits nearly 100% oral bioavailability, has minimal side effects and 90% of the of levetiracetam is excreted unchanged or as an inactive metabolite.

Preferred pyrrolidone compounds for use in the therapeutic methods of the invention induce at least about a 5% or 10% reduction in the occurrence of hyperkinetic movement disorder episodes, e.g., a reduction in the number of occurrences of repetitive involuntary movements or utterances, more preferably at least about a 15% or 20% reduction in the occurrence of hyperkinetic movement disorder episodes, and still more preferably induce at least a 25%, 30%, 40%, 50%, 60%, 70%, 80%, 90%, 95%, or 100% reduction in the occurrence of hyperkinetic movement disorder episodes.

In preferred embodiments, treatment methods of the invention induce a reduction of hyperkinetic movement disorder activity within 14 days of beginning administration of a pyrrolidone anti-convulsant compound. Preferably, the reduction in activity is induced within 10, 7, 6, 5, 4, or 3 days of beginning administration of the pyrrolidone compound. More preferably, reduction in activity is induced within 48, 36, 24 or 12 hours of commencing administration of the pyrrolidone compound.

In addition to the above discussed preferred pyrrolidone anti-convulsant compounds, suitable compounds for use in the methods of the invention are disclosed below. It should be appreciated however that the present invention is not limited by the particular pyrrolidone anti-convulsant compound, and the invention is applicable to any such pyrrolidone anti-convulsant compound now known or subsequently discovered or developed.

More specifically, suitable pyrrolidone anti-convulsant compounds for use in the methods of the invention include compounds of the following Formula I:

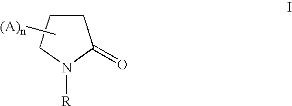

and pharmacologically acceptable salts thereof wherein

R is hydrogen, optionally substituted alkyl, optionally substituted alkenyl, optionally substituted alkynyl, optionally substituted, aralkyl, optionally substituted 2-acetamide, or optionally substituted aminoalkyl;

A is independently selected at each occurrence from the group consisting of hydrogen, optionally substituted alkyl, optionally substituted alkenyl, optionally substituted alkynyl, optionally substituted alkoxy, optionally substituted hydroxyalkyl, optionally substituted aminoalkyl, optionally substituted carbamoyl; hydroxy, amino, optionally substituted mono- or di-alkylamino; and

n is an integer of 0-6.

Preferred compounds of formula I include those compounds wherein

R is optionally substituted lower alkyl, optionally substituted benzyl, 2-acetamide groups which may be optionally substituted at N or β carbon with 0-4 lower alkyl groups;

A is hydrogen, optionally substituted lower alkyl, hydroxy, hydroxymethyl, amino, or optionally substituted aminoC₁₋₆alkyl; and

n is 0, 1 or 2.

Additional suitable compounds for use in the methods of the invention include. those of the following Formula II:

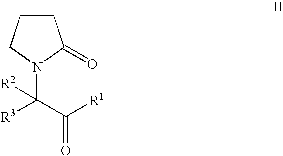

and pharmacologically acceptable salts thereof wherein

R¹ is optionally substituted alkyl, optionally substituted alkenyl, optionally substituted alkynyl, hydroxy, optionally substituted alkoxy, amino, optionally substituted mono- or di-alkylamino, or optionally substituted aminoalkyl; and

R² and R³ are independently selected from the group consisting of hydrogen, optionally substituted alkyl, optionally substituted alkenyl, optionally substituted alkynyl, optionally substituted alkoxy, optionally substituted hydroxyalkyl, optionally substituted aminoalkyl, optionally substituted carbamoyl; optionally substituted alkanoyl; hydroxy, amino, optionally substituted mono- or di-alkylamino; or

CR²R³ taken in combination are C═O or CH₂.

Preferred compounds of Formula II include those wherein

R¹ is hydroxy, amino, mono- or di-(C₁₋₆alkyl)amino, or C₁₋₆alkoxy;

R² is hydrogen or C₁₋₆alkyl; and

R³ is hydrogen.

Other preferred compounds which are suitable for use in the methods of the invention include those of the following Formula III:

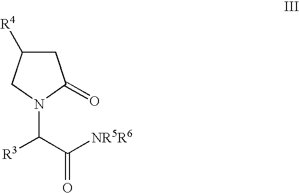

and pharmacologically acceptable salts thereof wherein

R² is hydrogen, optionally substituted alkyl, optionally substituted alkenyl, optionally substituted alkynyl, optionally substituted aralkyl, hydroxy, optionally substituted alkoxy, amino, optionally substituted mono- or di-alkylamino, or optionally substituted aminoalkyl;

R⁴ is hydrogen, hydroxy, amino, optionally substituted alkyl, optionally substituted alkenyl, optionally substituted alkynyl, optionally substituted alkoxy, optionally substituted hydroxyalkyl, optionally substituted aminoalkyl, or optionally substituted mono- or di-(alkyl)amino; and

R⁵ and R⁶ are independently selected from the group consisting of hydrogen, optionally substituted alkyl, optionally substituted alkenyl, optionally substituted alkynyl, optionally substituted hydroxyalkyl, or optionally substituted aminoalkyl.

Preferred compounds of Formula III include those wherein

R² is hydrogen or C₁₋₄alkyl;

R⁴ is hydrogen, hydroxy, hydroxymethyl or aminomethyl; and

R⁵ and R⁶ are independently selected from the group consisting of hydrogen and C₁₋₄alkyl.

Preferred compounds of Formula I which are suitable for use in the treatment methods of the invention include aniracetam (1-(4-Methoxy-benzoyl)-pyrrolidin-2-one), piracetam (2-(2-Oxo-pyrrolidin-1-yl)-acetamide), oxiracetam (2-(3-Hydroxy-2-oxo-pyrrolidin-1-yl)-acetamide), pramiracetam (1-[1-(2-{[(Diisopropylamino)-methyl]-amino}-acetyl)-vinyl]-pyrrolidin-2-one), nefiracetam, nebracetam (4-Aminomethyl-1-benzyl-pyrrolidin-2-one), fasoracetam ((R)-5-(Piperidine-1-carbonyl)-pyrrolidin-2-one), levetiracetam ((S)-2-(2-Oxo-pyrrolidin-1-yl)-butyramide). Particularly preferred compounds include pramiracetam and levetiracetam.

As discussed above, suitable pyrrolidone anti-convulsant compounds can be synthesized by known procedures. Some suitable inhibitor compounds also are commercially available, such as piracetam and levetiracetam, which may be purchased from UCP Pharma (Braine-1′Alleud, Belgium).

As also discussed above, typical subjects for administration in accordance with the invention are mammals, such as primates, especially humans.

Compounds of the invention are suitably administered to a subject in a water-soluble form, e.g., as a pharmaceutically acceptable salt of an organic or inorganic acid, e.g., hydrochloride, sulfate, hemi-sulfate, phosphate, nitrate, acetate, oxalate, citrate, maleate, mesylate, etc. Also, where an acidic group is present on an inhibitor compound, a pharmaceutically acceptable salt of an organic or inorganic base can be employed such as an ammonium salt, or salt of an organic amine, or a salt of an alkali metal or alkaline earth metal such as a potassium, calcium or sodium salt.

Specifically suitable pharmaceutically acceptable salts include those formed with a non-toxic cation, preferably an alkali metal cation such as K or Na, an alkaline earth metal cation such as Mg or Ca, another non-toxic metal cation such as Al or Zn or a non-toxic metalloid cation such as NH₄⁺, piperazinium or 2-hydroxyethylammonium. Certain preferred compounds suitable for use in the methods of the invention are sufficiently water soluble in neutral for such that the y may be delivered without pre-generation of a pharmaceutically acceptable salt.

Compounds suitable for use in the methods of the present invention include any and all different single pure isomers and mixtures of two or more isomers. The term isomers is intended to include diastereoisomers, enantiomers, regioisomers, structural isomers, rotational isomers, tautomers, and the like. For compounds which contain one or more stereogenic centers, e.g., chiral compounds, the methods of the invention may be carried out with a enantiomerically enriched compound, a racemate, or a mixture of diastereomers. Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral pyrrolidone compound is administered.

In the methods of the invention, a pyrrolidone anti-convulsant compound may be administered to a subject by a variety of routes including parenteral (including intravenous, subcutaneous, intramuscular and intradermal), topical (including buccal, sublingual), oral, nasal and the like.

Pyrrolidone anti-convulsant compounds for use in the methods of the invention can be employed, either alone or in combination with one or more other therapeutic agents, as a pharmaceutical composition in mixture with conventional excipient, i.e., pharmaceutically acceptable organic or inorganic carrier substances suitable for a desired route of administration which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parenteral application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc. Tablets, capsules and syrups or other fluids are generally preferred for oral administration.

A single or combination of more than one distinct pyrrolidone anti-convulsant compounds may be administered in a particular therapy. In this regard, a particular therapy can be optimized by selection of an optimal pyrrolidone anti-convulsant compound, or optimal “cocktail” of multiple pyrrolidone anti-convulsant compounds.

A pharmaceutical composition of the invention also may be packaged together with instructions (i.e. written, such as a written sheet) for treatment of a disorder as disclosed herein, e.g. instruction for treatment of a subject that is susceptible to or suffering from involuntary, repetitive movements or utterances, especially Tics, Tourette Syndrome, tremor, or myoclonus, even more preferably a subject that is susceptible to or suffering from tic disorders including simple tics, complex tics, and Tourette syndrome.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines. At least some pyrrolidone anti-convulsant compounds such as levetiracetam, piracetam and the like have been previously used clinically for treatment of patients suffering from epilepsy and thus safety of such compounds is established. Also, doses employed in such prior clinical applications will be provide further guidelines for preferred dosage amounts for methods of the present invention.

All documents mentioned herein are incorporated herein by reference.

The following non-limiting examples are illustrative of the invention.

### EXAMPLE 1

In one study, five patients suffering from tics, who previously failed to respond to standard drug therapies for tics, were administered with levetiracetam. Patients received either Low (250 mg BID) or standard doses (500 mg BID to 1500 mg BID) of levetiracetam. The majority of patients experienced a reduction of tic symptoms upon administration of levetiracetam.

### EXAMPLE 2

A patient suffering from Tourette syndrome is administered levetiractam. The patient is administered an initial dose of 250 mg, twice daily, which is increased over the course of 4 weeks to 500 mg, twice daily. Levetiractam may be administered indefinitely at a dosage of between 500 and 1000 mg, twice daily, in order to prevent onset of Tourette syndrome or to reduce the frequency of episodes of Tourette syndrome.

What is claimed is:

1. A method for treating a mammal suffering from or susceptible to a hyperkinetic movement disorder inducing involuntary, repetitive movements or utterances, comprising administering to the mammal a therapeutically effective amount of a pyrrolidone compound or a pharmaceutical composition of a pyrrolidone compound that has anticonvulsant activity.

2. The method of claim 1 wherein the mammal suffers from a tremor or tic disorder.

3. The method of claim 1, wherein the mammal suffers from a tremor.

4. The method of claim 3, wherein the mammal suffers from a resting tremor disorder, an action tremor disorder, an intention tremor disorder, or a tremor disorder induced by an underlying neurological or cerebellar disorder or disease.

5. The method of claim 4, wherein the mammal suffers from an idiopathic familial tremor associated with Parkinson's disease.

6. The method of claim 1, wherein the mammal suffers from a tic disorder.

7. The method of claim 6, wherein the mammal suffers from a simple tic disorder, a complex tic disorder, a multiple tic disorder, or Tourette syndrome.

8. The method of claim 7, wherein the mammal suffers from Tourette syndrome.

9. The method of any one of claims 1-8, wherein the pyrrolidone compound administered is a compound of the Formula I: 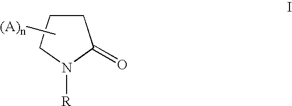
and pharmacologically acceptable salts thereof wherein
R is hydrogen, optionally substituted alkyl, optionally substituted alkenyl, optionally substituted alkynyl, optionally substituted, aralkyl, optionally substituted 2-acetamide, or optionally substituted aminoalkyl;
A is independently selected at each occurrence from the group consisting of hydrogen, optionally substituted alkyl, optionally substituted alkenyl, optionally substituted alkynyl, optionally substituted alkoxy, optionally substituted hydroxyalkyl, optionally substituted aminoalkyl, optionally substituted carbamoyl; hydroxy, amino, optionally substituted mono- or di-alkylamino; and
n is an integer of 0-6.

10. The method of claim 9, wherein the compound according to Formula I is a pyrrolidone compound wherein:
R is hydrogen, optionally substituted lower alkyl, optionally substituted benzyl, 2-acetamide groups which may be optionally substituted at N or β carbon with 0-4 lower alkyl groups;
A is hydrogen, optionally substituted lower alkyl, hydroxy, hydroxymethyl, amino, or optionally substituted aminoC₁₋₆alkyl; and
n is 0, 1 or 2.

11. The method of any one of claims 1-8, wherein the pyrrolidone compound administered is a compound of the Formula II: 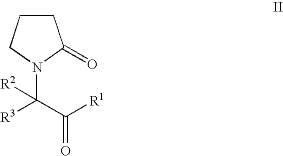
and pharmacologically acceptable salts thereof wherein
R¹ is optionally substituted alkyl, optionally substituted alkenyl, optionally substituted alkynyl, hydroxy, optionally substituted alkoxy, amino, optionally substituted mono- or di-alkylamino, or optionally substituted aminoalkyl; and
R² and R³ are independently selected from the group consisting of hydrogen, optionally substituted alkyl, optionally substituted alkenyl, optionally substituted alkynyl, optionally substituted alkoxy, optionally substituted hydroxyalkyl, optionally substituted aminoalkyl, optionally substituted carbamoyl; optionally substituted alkanoyl; hydroxy, amino, optionally substituted mono- or di-alkylamino; or
CR²R³ taken in combination are C═O or CH₂.

12. The method of any one of claims 1-8, wherein the pyrrolidone compound administered is a compound of the Formula III: 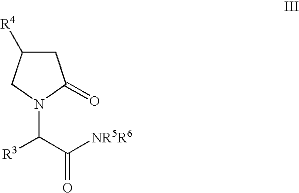
and pharmacologically acceptable salts thereof wherein
R² is hydrogen, optionally substituted alkyl, optionally substituted alkenyl, optionally substituted alkynyl, optionally substituted aralkyl, hydroxy, optionally substituted alkoxy, amino, optionally substituted mono- or di-alkylamino, or optionally substituted aminoalkyl;
R⁴ is hydrogen, hydroxy, amino, optionally substituted alkyl, optionally substituted alkenyl, optionally substituted alkynyl, optionally substituted alkoxy, optionally substituted hydroxyalkyl, optionally substituted aminoalkyl, or optionally substituted mono- or di-(alkyl)amino; and
R⁵ and R⁶ are independently selected from the group consisting of hydrogen, optionally substituted alkyl, optionally substituted alkenyl, optionally substituted alkynyl, optionally substituted hydroxyalkyl, or optionally substituted aminoalkyl.

13. The method of claim 12, wherein the compound of Formula III is a pyrrolidone compound wherein
R² is hydrogen or C₁₋₄alkyl;
R⁴ is hydrogen, hydroxy, hydroxymethyl or aminomethyl; and
R⁵ and R⁶ are independently selected from the group consisting of hydrogen and C₁₋₄alkyl.

14. The method of any one of claims 1-8, wherein the pyrrolidone compound administered is a compound selected from the group consisting of aniracetam, piracetam, oxiracetam, pramiracetam, nefiracetam, nebracetam, fasoracetam, and levetiracetam.

15. The method of any one of claims 1-8, wherein the pyrrolidone compound administered is pramiracetam, piracetam, or levetiracetam.

16. The method of any one of claims 1-8, wherein the pyrrolidone compound administered is levetiracetam.

17. The method of any one of claims 1-8, wherein the pyrrolidone compound administered is piracetam.

18. The method of any one of claims 1 through 17 wherein the mammal has been identified and selected for treatment of a hyperkinetic movement disorder inducing involuntary, repetitive movements or utterances, and the pyrrolidone compound is then administered to the identified and selected mammal.

19. A method for treating a mammal suffering from or susceptible to a tic disorder, comprising administering to the mammal a therapeutically effective amount of levetiractam or piracetam.

20. The method of claim 19 wherein the tic disorder is a complex tic, multiple tic or Tourette syndrome where the mammal suffers from involuntary repetitive movements or vocalizations.

21. The method of claim 19 or 20 wherein the mammal have been identified and selected form treatment for a tic disorder, and the levetiractam or piracetam is administered to the identified and selected mammal.

22. A method of reducing, preventing or delaying onset of a tic disorder comprising administering an effective amount of levetiractam to a patient.

23. The method of claim 20, wherein the tic disorder is Tourette syndrome.

24. A method of any one of claims 1 through 23, wherein the pyrrolidone compound induces at least about a 10 percent reduction in the frequency, duration or severity of occurrences of the hyperkinetic movement disorder.

25. A method of any one of claims 1 through 23, wherein the pyrrolidone compound induces at least about a 25 percent reduction in the frequency, duration or severity of occurrences of the hyperkinetic movement disorder.

26. A method of any one of claims 1 through 23, wherein the pyrrolidone compound induces at least about a 50 percent reduction in the frequency, duration or severity of occurrences of the hyperkinetic movement disorder.

27. A method of any one of claims 1 through 23, wherein the pyrrolidone compound induces at least about a 80 percent reduction in the frequency, duration or severity of occurrences of the hyperkinetic movement disorder.

28. A method of any one of claims 1 through 23, wherein the pyrrolidone compound induces at least about a 90 percent reduction in the frequency, duration or severity of occurrences of the hyperkinetic movement disorder.

29. A method of any one of claims 1 through 23, wherein the pyrrolidone compound reduces the frequency of onset of involuntary, repetitive movements or utterances associated with a hyperkinetic movement disorder within 7 days of commencing the treatment method.

30. The method of claim 29, wherein the reduction of onset of involuntary, repetitive movements or utterances occurs within 3 days of commencing the treatment method.

31. The method of claim 29, wherein the reduction of onset of involuntary, repetitive movements or utterances occurs within 24 hours of commencing the treatment method.

32. The method of any one of claims 1 through 31, wherein the compound is administered to a primate.

33. The method of any one of claims 1 through 29, wherein the compound is administered to a human.

34. A package comprising a pharmaceutical composition of a pyrrolidone compound that has anticonvulsant activity in a container and further comprising indicia comprising instructions for using the composition to treat a patient suffering from an hyperkinetic movement disorder.

35. A package comprising a pharmaceutical composition of claim 34 in a container and further comprising indicia comprising at least one of:
instructions for using the composition to treat a patient suffering from a tic disorder,
instructions for using the composition to treat a patient suffering from a multiple tic disorder or Tourette syndrome, or
instructions for using the composition to treat a patient suffering from a tremor.
